(19) United States
(12) Patent Application Publication
(10) Pub. No.: US 2023/0277274 A1
(43) Pub. Date: Sep. 7, 2023

(54) SURGICAL AUDIO AND VIDEO COLLECTION DEVICE

(71) Applicant: Bill Yang, Taipei City (TW)

(72) Inventor: Bill Yang, Taipei City (TW)

(73) Assignee: Cotron Corporation, Taipei City (TW)

(21) Appl. No.: 17/889,383

(22) Filed: Aug. 16, 2022

Related U.S. Application Data
(63) Continuation-in-part of application No. 17/745,772, filed on May 16, 2022, now abandoned.

(30) Foreign Application Priority Data
Mar. 4, 2022 (TW) 111202154
Jul. 13, 2022 (TW) 111207482

(51) Int. Cl. A61B 90/00; A61B 90/50

(52) U.S. Cl. CPC A61B 90/361; A61B 90/50; A61B 2090/502

(57) ABSTRACT

A surgical audio and video collection device includes a processor, a video camera and a skin-touch vibration pickup. The video camera electrically connected to the processor is used to be worn on the body of the surgical operator and record video. The skin-touch vibration pickup is used to be attached on the skin of the surgical operator and sense the vibration of the skin to pick up the sound. The skin-touch vibration pickup is signally connected to the processor to transmit voice message to the processor. The voice message includes at least one of voice commands for controlling the video camera and voice tags for being added to the video recorded by the video camera.

### CROSS REFERENCE TO RELATED APPLICATION

This application is a continuation-in-part application of and claims the priority benefit of U.S. Application Ser. No. 17/745,772, filed on May 16, 2022, now pending, which claims the priority benefit of Taiwan application serial no. 111202154, filed on Mar. 4, 2022. This application also claims the priority benefit of Taiwan application serial no. 111207482, filed on Jul. 13, 2022. The entirety of each of the above-mentioned patent applications is hereby incorporated by reference herein and made a part of specification.

### BACKGROUND

### Field of the Disclosure

The present disclosure relates to an audio and video collection device, and in particular, a surgical audio and video collection device.

### Description of Related Art

The development of technology has made the collection of surgical audio and video easier. Surgical video may provide a record of an operation scene, and has been adopted as an important means for learning, teaching and academic activities among medical personnel. Surgical video helps resident physicians and researchers to learn the details of surgery and observe the actual operation of surgical instruments.

Based on the relationship between the video camera, the surgical equipment and the surgical operator, the surgical audio and video collection device is divided into the following three categories: non-integrated, operator-integrated and equipment-integrated. Among them, the operator-integrated type of surgical audio and video collection device mainly performs video recording by fixing the video camera on the body of the surgical operator, thereby providing surgical audio and video from the first perspective.

### SUMMARY OF THE DISCLOSURE

The present disclosure provides a surgical audio and video collection device, which may solve existing defects.

A surgical audio and video collection device of the disclosure includes a processor, a video camera and a skin-touch vibration pickup. The video camera electrically connected to the processor is configured to be worn on the body of the surgical operator and record video. The skin-touch vibration pickup is configured to be attached on the skin of the surgical operator and sense the vibration of the skin to pick up the sound. The skin-touch vibration pickup is signally connected to the processor to transmit voice message to the processor. The voice message includes at least one of voice commands for controlling the video camera and voice tags for being added to the video recorded by the video camera.

In an embodiment of the disclosure, the surgical audio and video collection device further includes a headband, and the video camera is mounted on the headband.

In an embodiment of the present disclosure, the skin-touch vibration pickup is mounted on the headband and configured to contact the head of the surgical operator.

In an embodiment of the disclosure, the surgical audio and video collection device further includes a neckband, and the skin-touch vibration pickup is mounted on the neckband and configured to contact the neck of the surgical operator.

In an embodiment of the disclosure, the surgical audio and video collection device further includes an earphone, which is configured to be worn on or around the ear of the surgical operator, to receive an external sound source and transmit the external sound source to the ear of the surgical operator.

In an embodiment of the present disclosure, the earphone is an osteoconductive earphone.

In an embodiment of the disclosure, the surgical audio and video collection device further includes a host, and the host has a processor, and the earphone receives an external sound source from the host.

In an embodiment of the disclosure, the surgical audio and video collection device further includes a host, and the host has a processor, the video camera transmits the video to the host, and the skin-touch vibration pickup transmits voice commands to the video camera through the host.

In an embodiment of the disclosure, the skin-touch vibration pickup transmits voice messages externally through the host.

In an embodiment of the disclosure, the surgical audio and video collection device further includes a high-frequency microphone, which is electrically connected to the host.

In an embodiment of the disclosure, the surgical audio and video collection device further includes a wireless transceiver. The video camera is electrically connected to the wireless transceiver and transmits the video to the host through the wireless transceiver. The skin-touch vibration pickup is electrically connected to the wireless transceiver and transmits voice messages to the host through the wireless transceiver.

In an embodiment of the disclosure, the surgical audio and video collection device further includes a host, and the host has a processor. The video camera transmits the video to the host, and the skin-touch vibration pickup transmits a voice tag to the host.

In an embodiment of the disclosure, the surgical audio and video collection device further includes a high-frequency microphone, which is electrically connected to the processor.

Based on the above, in the surgical audio and video collection device of the present disclosure, the surgical operator may voice control the video camera or add voice tags to the video. Therefore, the surgical audio and video collection device of the present disclosure has the advantage of being easy to control.

### BRIEF DESCRIPTION OF THE DRAWINGS

FIG. 1 is a schematic view of a surgical audio and video collection device according to an embodiment of the present disclosure.

FIG. 2 is a schematic view of a surgical audio and video collection device according to another embodiment of the present disclosure.

FIG. 3 is a schematic view of a surgical audio and video collection device according to still another embodiment of the present disclosure.

### DESCRIPTION OF EMBODIMENTS

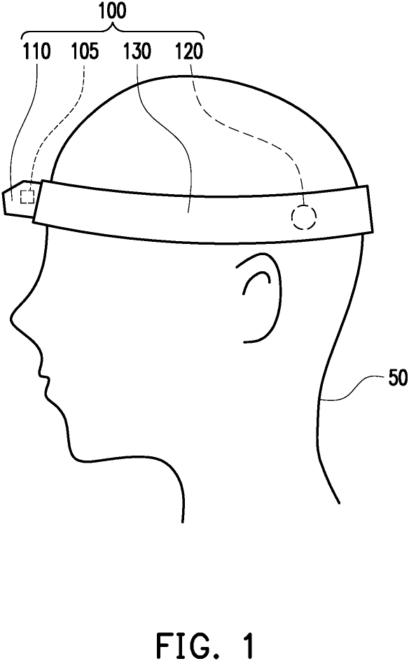

FIG. 1 is a schematic view of a surgical audio and video collection device according to an embodiment of the present disclosure. Please refer to FIG. 1, the surgical audio and video collection device 100 of this embodiment includes a processor 105, a video camera 110 and a skin-touch vibration pickup 120. The video camera 110 is configured to be worn on the body of the surgical operator 50 and record video. The skin-touch vibration pickup 120 electrically connected to the processor 105 is configured to be attached to the skin of the surgical operator 50 and sense the vibration of the skin to pick up sound. The skin-touch vibration pickup 120 is signally connected to the processor 105 to transmit a voice message to the processor 105. Here, the skin-touch vibration pickup 120 being signally connected to the processor 105 means that the skin-touch vibration pickup 120 may transmit signals to the processor 105, but it is not limited that the signals are transmitted in a wired or wireless manner.

In the surgical audio and video collection device 100 of this embodiment, the skin-touch vibration pickup 120 is configured to pick up sound. That is, unlike the general microphone that senses air vibration caused by the sound, the skin-touch vibration pickup 120 senses the vibration generated by the skin attached thereto when the surgical operator 50 speaks. Therefore, even in a situation where the ambient sound of the operation scene is noisy, the content of speech delivered by the surgical operator 50 may still be accurately received by the skin-touch vibration pickup 120. Thus, the processor 105 may receive voice messages from the skin-touch vibration pickup 120.

When the processor 105 determines that the voice message includes a voice command for the video camera 110, the processor 105 transmits the voice command to the video camera 110, and the video camera 110 starts video recording, pauses video recording, ends video recording or perform other actions according to the commands. In this way, not only can the surgical operator 50 have more control over the video camera 110, but also there is no need to set foot pedals that become obstacles for the surgical operator.

On the other hand, the voice message may further include a voice tag in the video recorded by the video camera 110. For example, when the processor 105 determines that the voice message contains preset keywords, such as “here is the key point”, “this is the beginning of the key point”, “this is the end of the key point”, etc., a tag is added at the corresponding section in the video recorded by the video camera 110. In this way, when viewing the video recorded by the video camera 110 in the future, it is easy to quickly find the section of the key segment, so as to improve the practicability of the video. The preset keywords described herein may be built-in in the system, but may also be set by users to improve flexibility.

In this embodiment, the surgical audio and video collection device 100 further includes a headband 130, and the video camera 110 is mounted on the headband 130. The headband 130 may be an elastic band, or a belt whose length is adjusted by using buttons, velcro tape or other methods, or a steel ring or other suitable forms. In this embodiment, the skin-touch vibration pickup 120 is mounted on the headband 130 and configured to contact the head of the surgical operator 50. In other words, the headband 130 may provide an appropriate force to press the skin-touch vibration pickup 120 against the head of the surgical operator 50. Here, although it is described that the skin-touch vibration pickup 120 contacts the head of the surgical operator 50, the two are not limited to be in direct contact, and there may be surgical caps or other objects arranged between the two, as long as the skin-touch vibration pickup 120 can pick up sound correctly.

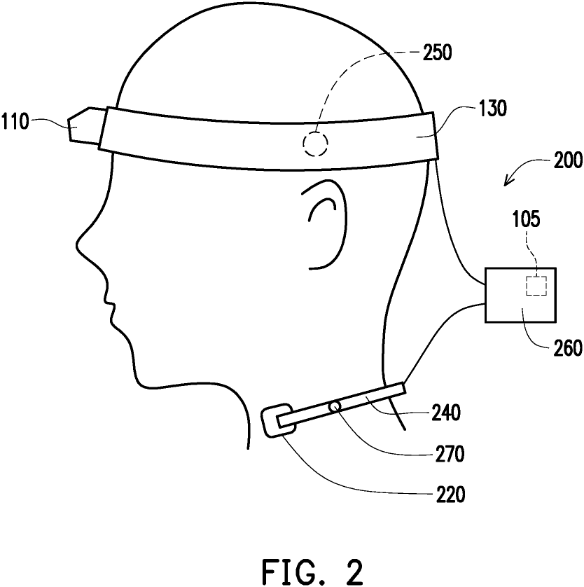

FIG. 2 is a schematic view of a surgical audio and video collection device according to another embodiment of the present disclosure. Referring to FIG. 2, the surgical audio and video collection device 200 of this embodiment is similar to the surgical audio and video collection device 100 of FIG. 1, and only the differences between the two are described here. In this embodiment, the surgical audio and video collection device 200 further includes a neckband 240. The skin-touch vibration pickup 220 of this embodiment is mounted on the neckband 240 and configured to contact the neck of the surgical operator 50. In other words, the neckband 240 may provide an appropriate force to press the skin-touch vibration pickup 220 against the neck of the surgical operator 50. The neckband 240 may be an elastic band, or a belt whose length is adjusted by using buttons, velcro tape or other methods, or a steel ring or other suitable forms. In other embodiments, the skin-touch vibration pickup 220 may also be attached to the skin of the surgical operator 50 with breathable tape.

In this embodiment, the skin-touch vibration pickup 220 is in contact with the neck of the surgical operator 50. Because the head of the surgical operator 50 normally wears a surgical cap and the neck is normally bare, the skin-touch vibration pickup 220 is more likely to be in direct contact the skin of the neck, thereby obtaining a better sound pickup effect.

In the present embodiment, the surgical audio and video collection device 200 further includes an earphone 250 configured to be worn on or around the ears of the surgical operator 50 to receive external sound sources and transmit external sound sources to the ears of the surgical operator 50. For example, the earphone 250 may communicate with a mobile phone or other devices, so the surgical operator 50 may receive the voice of the personnel outside the operating room through the earphone 250. In this embodiment, the earphone 250 may be an osteoconductive earphone. Therefore, the earphone 250 may transmit sound to the ears of the surgical operator 50, and the ears of the surgical operator 50 may still receive the sounds of personnel and equipment in the operating room.

In this embodiment, the surgical audio and video collection device 200 further includes a host 260. The host 260 has a processor 105. The earphone 250 receives an external sound source from the host 260. Communication between the earphone 250 and the host 260 may be performed in a wired or wireless manner.

In this embodiment, the video camera 110 may transmit video to the host 260, and the host 260 stores and/or transmits the video. In the embodiment shown in FIG. 1, the processor 105 and the video camera 110 are arranged in the same housing, and may store and/or transmit videos by themselves, but the volume thereof may be relatively large. Communication between the video camera 110 and the host 260 may be performed in a wired or wireless manner, such as using Wifi, Bluetooth or other wireless communication protocols. The skin-touch vibration pickup 220 transmits a voice command to the video camera 110 through the processor 105 of the host 260. Communication between the skin-touch vibration pickup 220 and the processor 105 of the host 260 may be performed in a wired or wireless manner. The voice message recorded by the skin-touch vibration pickup 220 may be converted into a control command by the processor 105 of the host 260 and then transmitted to the video camera 110. In other embodiments, the processor 105 may also be a general term for multiple processors. For example, the host 260 has one of the multiple processors collectively referred to as the processor 105, and the video camera 110 also includes another of the multiple processors collectively referred to as the processor 105. Therefore, the voice message recorded by the skin-touch vibration pickup 220 may also be directly transmitted to the processor included in the video camera 110 to be converted into control commands and executed.

On the other hand, the voice message recorded by the skin-touch vibration pickup 220 may be determined by the processor 105 of the host 260 whether the voice message contains a preset keyword. When it is determined that the voice message contains a preset keyword, a tag is added at the corresponding section in the video recorded by the video camera 110. However, the voice message recorded by the skin-touch vibration pickup 220 may also be directly transmitted to the video camera 110, and the processor in the video camera 110 determines whether the preset keyword is included. When it is determined that the voice message contains the preset key, a tag is added at the corresponding section in the video recorded by the video camera 110.

In this embodiment, the skin-touch vibration pickup 220 may further transmit voice messages externally through the host 260. In addition, the earphone 250 may receive an external sound source through the host 260 and transmit the external sound source to the ears of the surgical operator 50. Therefore, the surgical operator 50 uses the skin-touch vibration pickup 220 and the earphone 250 to communicate with the personnel outside the operating room through the host 260.

In this embodiment, the surgical audio and video collection device 200 further includes a high-frequency microphone 270, which is electrically connected to the processor 105 of the host 260. For example, the high-frequency microphone 270 may include a microphone and a filter. After the microphone picks up the sound, the low-frequency part is filtered out by the filter and the high-frequency part is retained. The skin-touch vibration pickup 220 mainly picks up the low-frequency part of the sound, while the high-frequency microphone 270 may pick up the high-frequency part of the sound. In this way, it is possible to avoid recording the ambient noise, and the high-frequency part of the sound such as breath sounds emitted by the surgical operator 50 may be supplemented. Therefore, the accuracy of the recognition of the voice message may be improved, and when the surgical operator 50 carries out communication through the host 260, the sound transmitted to the outside has better fidelity.

In other embodiments, the high-frequency microphone 270 of the surgical audio and video collection device may also be electrically connected to the processor of the video camera 110, and the audio recording results thereof may be used for voice-controlling the video camera 110 or stored by the processor of the video camera 110.

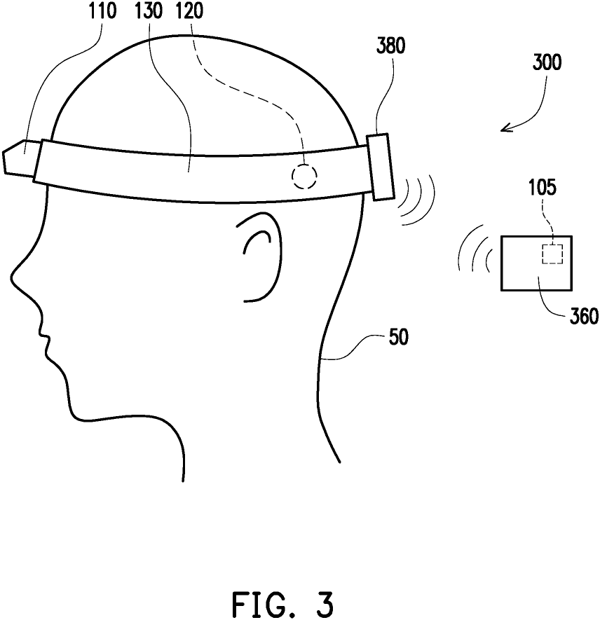

FIG. 3 is a schematic view of a surgical audio and video collection device according to yet another embodiment of the disclosure. Please refer to FIG. 3, the surgical audio and video collection device 300 of this embodiment is similar to the surgical audio and video collection device 100 of FIG. 1, and only the differences between the two are described here. In this embodiment, the surgical audio and video collection device 300 further includes a host 360 and a wireless transceiver 380. The video camera 110 is electrically connected to the wireless transceiver 380, and the host 360 is signally connected to the wireless transceiver 380 in a wireless manner, such as using Wifi, Bluetooth or other wireless communication protocols. Therefore, the video camera 110 may transmit the video to the host 360 through the wireless transceiver 380. The skin-touch vibration pickup 120 is electrically connected to the wireless transceiver 380, and transmits the recorded voice messages to the host 360 through the wireless transceiver 380. The processor 105 of the host 360 converts the received voice messages into control commands and sends the control commands back to the transceiver 380 wirelessly, and then the wireless transceiver 380 transmits the control commands to the video camera 110. Or, when the processor 105 of the host 360 determines that the voice message contains a voice tag, such as preset keywords, which may be “here is the key point”, “this is the beginning of the key point”, “this is the end of the key point”, etc., a tag is added at the corresponding section in the video recorded by the video camera 110.

To sum up, in the surgical audio and video collection device of the present disclosure, the skin-touch vibration pickup senses the vibration of the skin of the surgical operator to pick up sound. Therefore, the surgical operator may easily control the surgical audio and video collection device or add voice tags to the recorded video.

What is claimed is:

1. A surgical audio and video collection device, comprising:
a processor;
a video camera, configured to be worn on a body of a surgical operator and record a video, and electrically connected to the processor; and
a skin-touch vibration pickup, configured to be attached on a skin of the surgical operator and sense a vibration of the skin to pick up a sound, wherein the skin-touch vibration pickup is signally connected to the processor to transmit a voice message to the processor, the voice message comprises at least one of a voice command for controlling the video camera and a voice tag for being added to the video recorded by the video camera.

2. The surgical audio and video collection device according to claim 1, further comprising a headband, wherein the video camera is mounted on the headband.

3. The surgical audio and video collection device according to claim 2, wherein the skin-touch vibration pickup is mounted on the headband and configured to contact a head of the surgical operator.

4. The surgical audio and video collection device according to claim 1, further comprising a neckband, wherein the skin-touch vibration pickup is mounted on the neckband and configured to contact a neck of the surgical operator.

5. The surgical audio and video collection device according to claim 1, further comprising an earphone, which is configured to be worn on or around an ear of the surgical operator, to receive an external sound source and transmit the external sound source to the ear of the surgical operator.

6. The surgical audio and video collection device according to claim 5, wherein the earphone is an osteoconductive earphone.

7. The surgical audio and video collection device according to claim 5, further comprising a host, wherein the host has the processor, and the earphone receives the external sound source from the host.

8. The surgical audio and video collection device according to claim 1, further comprising a host, wherein the host has the processor, the video camera transmits the video to the host, and the skin-touch vibration pickup transmits the voice commands to the video camera through the host.

9. The surgical audio and video collection device according to claim 8, wherein the skin-touch vibration pickup transmits the voice message externally through the host.

10. The surgical audio and video collection device according to claim 8, further comprising a high-frequency microphone, which is electrically connected to the host.

11. The surgical audio and video collection device according to claim 8, further comprising a wireless transceiver, wherein the video camera is electrically connected to the wireless transceiver and transmits the video to the host through the wireless transceiver, the skin-touch vibration pickup is electrically connected to the wireless transceiver and transmits the voice message to the host through the wireless transceiver.

12. The surgical audio and video collection device according to claim 1, further comprising a host, wherein the host has the processor, the video camera transmits the video to the host, and the skin-touch vibration pickup transmits the voice tag to the host.

13. The surgical audio and video collection device according to claim 1, further comprising a high-frequency microphone, which is electrically connected to the processor.
